(19) United States
(12) Patent Application Publication
(10) Pub. No.: US 2011/0124986 A1
(43) Pub. Date: May 26, 2011

(54) Microchip, Blood Analysis System and Blood Analysis Method

(76) Inventors: Masaaki Takama, Tokyo (JP); Yoshiaki Hirahara, Tokyo (JP)

(21) Appl. No.: 13/054,990

(22) PCT Filed: Jul. 6, 2009
(86) PCT No.: PCT/JP2009/062282

§ 371 Date: Jan. 20, 2011

(30) Foreign Application Priority Data
Jul. 23, 2008 (JP) 2008-189494
Nov. 7, 2008 (JP) 2008-286318

(51) Int. Cl. A61B 5/00

(52) U.S. Cl. 600/309

(57) ABSTRACT

A microchip to be installed in a blood analysis system for measuring properties of blood, provided with a plurality of channels through which blood passes, wherein only each of first subset channels among the plurality of channels is provided with a first plurality of barriers for locally changing a direction of blood flow, in a blood flow direction on a wall face demarcating the channels, and among the first plurality of barriers, barriers which are adjacent to each other in the blood flow direction differ in shape from each other.

### TECHNICAL FIELD

The present invention relates to a microchip, a blood analysis system and a blood analysis method.

### BACKGROUND TECHNOLOGY

In recent years, along with increasing awareness of health, blood properties such as blood fluidity and deformability of blood cell has been paid attention as a health barometer. For example, the fluidity is also called as the degree of smoothness, and it means that the higher the fluidity or the smoothness is the better in health.

As a blood analysis apparatus for investigating the above blood properties, it has been known that blood or blood cells are allowed to pass through a channel of fine groove, and properties of the blood are numerically measured (please refer for example Patent Documents 1 to 6).

Among those apparatuses, according to the blood analysis apparatus described in Patent Documents 1 or 2, from a view point of generalizing the result of analysis by eliminating fluctuations by each analysis, the blood properties are analyzed with the apparatus where a plurality of fine channels arranged in an array.

Further, according to the blood analysis apparatus described in Patent Documents 3 or 4, from a view point of accurately quantifying the blood properties by simulating the blood flow to that in a blood vessel of human body, shapes of the channels are made to have variations. More specifically, in the apparatus of Patent Document 3, a channel width in a part of the channels is narrowed in the blood flow direction, and in the apparatus of Patent Document 4, a plurality of barriers are arranged in a part of the channels.

Meanwhile, in a human body, barriers in the blood vessel may be changed in the blood flow direction by collisions of the blood, even in cases where a plurality of barriers with an identical size and shape are provided.

### Prior Art Document

### Patent Document

### Patent Document 1: Unexamined Japanese Patent Application Publication No. 2006-145345

### Patent Document 2: Unexamined Japanese Patent Application Publication No. 2005-265634

Patent Document 3: Unexamined Japanese Patent Application Publication No. H02-130471

### Patent Document 4: Unexamined Japanese Patent Application Publication No. 2004-4002

### Patent Document 5: Japanese Translation of PCT International Application Publication No. 2006-501449

### Patent Document 6: Unexamined Japanese Patent Application Publication No. 2005-164296

### SUMMARY OF THE INVENTION

### Problems to be Solved by the Invention

However, in the apparatus of the above Patent Document 3, the bathers in the channel have the same shape in the blood flow direction (adjacent bathers in the blood flow direction have the same shape with each other), which does not fully reproduce the complex human body. Therefore, the accurate blood analysis simulating inside the blood vessel cannot be performed.

The present invention is accomplished in view of the above situations, to proved a microchip, a blood analysis system and a blood analysis method that enables the blood analysis by more accurately simulating the inside of the blood vessel than in the existing method.

The invention described in claim 1 is a microchip to be equipped in a blood analysis system for measuring a blood property, characterized in that:

the microchip comprises a plurality of channels through which blood passes, wherein only each of first subset channels among the plurality of channels is provided with a first plurality of barriers for locally changing a direction of blood flow, in a blood flow direction on a wall face demarcating the channels, and among the first plurality of barriers, bathers which are adjacent to each other in the blood flow direction differ in shape from each other.

The invention described in claim 2 is a microchip to be equipped in a blood analysis system for measuring a blood property, characterized in that:

the microchip comprises a plurality of channels through which blood passes, wherein only each of first subset channels among the plurality of channels is provided with a second plurality of bathers for locally changing a direction of blood flow, in a blood flow direction on a wall face demarcating the channels, and the second plurality of barriers have random shapes.

The invention described in claim 3 is a microchip described in claim 1 or 2, characterized in that:

each of other subset channels than the first subset channels among the plurality of channels is provided with a third plurality of barriers for locally changing a direction of blood flow, in a blood flow direction on a wall face demarcating the channels with a constant interval, and the third plurality of barriers have a same shape.

The invention described in claim 4 is a microchip described in any one of claims 1 to 3, characterized in that:

the plurality of barriers are provided so as to protrude inside the channels.

The invention described in claim 5 is a microchip described in any one of claims 1 to 4, characterized in that:

an inner wall portion of the plurality of channels is configured to have a cross section area becoming wider or becoming narrower from upstream toward downstream of the blood flow.

The invention described in claim 6 is a microchip described in any one of claims 1 to 5, characterized in that:

the wall face demarcating the channels, on which the plurality of barriers are not provided, is formed flat.

The invention described in claim 7 is a microchip described in any one of claims 1 to 6, characterized in that:

the wall face demarcating the plurality of channels has a circular section.

The invention described in claim 8 is a blood analysis system characterizing in being provided with: a microchip described in any one of claims 1 to 7;

an imaging device to capture an image of blood flow in the plurality of channels of the microchip; and an analyzing section to analyze the image captured by the imaging device and calculates a blood property.

The invention described in claim 9 is the blood analysis system described in claim 8, characterized in that: the analyzing section calculates a blood property by analyzing the captured image at a prescribed channel among the plurality of channels.

The invention described in claim 10 is the blood analysis system described in claim 8, characterized in that: the analyzing section compares each blood properties by analyzing each of the captured images at a channel of the first subset channels and at a channel of other channels than the first subset channels among the plurality of channels respectively.

The invention described in claim 11 is the blood analysis system described in any one of claims 8 to 10, characterized in that: the imaging device captures images of blood flow at a channel of the first subset channels and at the other channel among the plurality of channels.

The invention described in claim 12 is a blood analysis method utilizing the microchip described in any one of claims 1 to 7, the method being characterized in including: an imaging process to capture an image of blood flow in the plurality of channels of the microchip; and an analyzing process to analyze the image captured by the imaging device and calculate a blood property.

The invention described in claim 13 is the blood analysis method described in claim 12, characterized in that the analyzing process calculates a blood property by analyzing the captured image at a prescribed channel among the plurality of channels.

The invention described in claim 14 is the blood analysis method described in claim 12, characterized in that: the analyzing process compares each blood properties by analyzing each of the captured images at a channel of the first subset channels and at a channel of other channels than the first subset channels among the plurality of channels respectively.

The invention described in claim 15 is the blood analysis method described in any one of claims 12 to 14, characterized in that: the imaging process captures images of blood flow at a channel of the first subset channels and at a channel of other channels than the first subset channels among the plurality of channels.

### Effect of the Invention

According to the invention described in claim 1, since only each of first subset channels among the plurality of channels is provided with a first plurality of barriers for locally changing the direction of the blood flow in the blood flow direction on the wall face demarcating the channel, the shapes of channels are made different between said first subset channels and the other channels. Therefore, by comparing the blood vessels with barriers and the blood vessels without barriers, blood properties can be exactly analyzed.

Further, among the first plurality of bathers, barriers which are adjacent to each other in the blood flow direction are made different in shape from each other. This is for simulating to blood vessel condition of human body by structuring in different shapes with each other, since plurality of barriers generated in human body (such as thromboses) do not maintain same shapes and may move or change in shape by new blood flows. According to this configuration, compared to the case where barriers which are adjacent to each other in the blood flow direction are made in same shape, the blood flow is made closer to the condition in the blood vessel of the human body for analyzing the blood properties.

Therefore, compared to the conventional cases, the blood property analysis more exactly simulating inside the blood vessel is enabled.

According to the invention described in claim 2, since first subset channels among the plurality of channels is provided with a second plurality of barriers for locally changing the direction of the blood flow in the blood flow direction on the wall face demarcating the channel, the shapes of channels are made different between said first subset channels and the other channels. Therefore, by comparing the blood vessels with barriers and the blood vessels without barriers, blood properties can be exactly analyzed.

Further, the second plurality of barriers is configured to have random shapes. This is for simulating to blood vessel condition of human body by structuring in random shapes, since plurality of barriers generated in human body (such as thromboses) do not maintain same shapes and may move or change in shape by new blood flows. According to this configuration, compared to the case where barriers are made regularly in shape, the blood flow is made closer to the condition in the blood vessel of the human body for analyzing the blood properties.

Therefore, compared to the conventional cases, the blood property analysis more precisely simulating inside the blood vessel is enabled.

According to the invention described in claim 3, each of other subset channels than the first subset channels among the plurality of channels is provided with a third plurality of barriers for locally changing the direction of the blood flow in the blood flow direction on the wall face demarcating the channel with a constant interval, and the third plurality of bathers have the same shape. Therefore, the blood flow can be made nearer in condition where thromboses are made in the blood vessel, and further changing of blood flow by the thromboses can be observed.

According to the invention described in claim 4, since the plurality of barriers are provided to be protruded inside the channels, the blood flow can be made closer to the condition inside the blood vessel to perform the blood analysis.

According to the invention described in claim 5, an inner wall of the plurality of channels is configured to have a cross section area becoming wider or becoming narrower from upstream toward down stream of blood flow. Therefore, the blood flow can be made closer in condition of the blood vessel, and blood properties can be more exactly analyzed by simulating inside the blood vessel.

According to the invention described in claim 6, since among the plurality of channels the wall face demarcating the channel on which the plurality of barriers are not provided is formed flat, the blood flow can be made closer to condition of normal blood vessel. Therefore, by comparing the blood vessels with barriers and the blood vessels without barriers, blood properties can be exactly analyzed.

According to the invention described in claim 7, since, the wall face demarcating the plurality of channels has a circular cross-section, the blood flow can be made closer to the condition inside the blood vessel to perform the blood analysis.

According to the invention described in claims 8 or 12, since an image of the blood flow in the plurality of channels in the microchip is captured and the image captured by the imaging device is analyzed to calculate a blood property, the condition of the blood flowing in the microchip is image captured and the calculation of the blood property is enabled.

According to the invention described in claims 9 and 13, since the captured image at a prescribed channel among the plurality of channels is analyzed to calculate a blood property, the analysis is made easier compared to the case where captured images for each channels are analyzed to calculate the blood property.

According to the invention described in claims 10 or 14, since each blood properties is calculated by analyzing each of the captured images at a channel of the first subset channels and at a channel of other channels than the first subset channels among the plurality of channels, and the each blood properties is compared, the blood property analysis can be performed in such cases as where thromboses or atherosclerosis are made in the blood vessel.

According to the invention described in claims 11 and 15, since the imaging device captures images of blood flow at a prescribed channel and the other channel among the plurality of channels, the blood property analysis can be made easier compared to the cases where either one of them is Image captured.

### BRIEF DESCRIPTION OF THE DRAWINGS

FIG. 1 is a block diagram showing a total configuration of a blood analysis system relating to the present invention.

FIGS. 2a-2c are drawings showing a microchip, where FIG. 2a is a plan view, FIG. 2b is an exploded side view, and FIG. 2c is a partially enlarged view.

FIGS. 3a and 3b are drawings to explain the channel of the microchip.

FIG. 4 is a plan view to explain the channel of the microchip.

FIG. 5 is a plan view to explain the channel of the microchip.

FIG. 6 is a conceptual drawing to explain the channel of the microchip.

### EMBODIMENTS FOR ENFORCING THE INVENTION

Referring to the drawings, embodiments of the present invention will be described below.

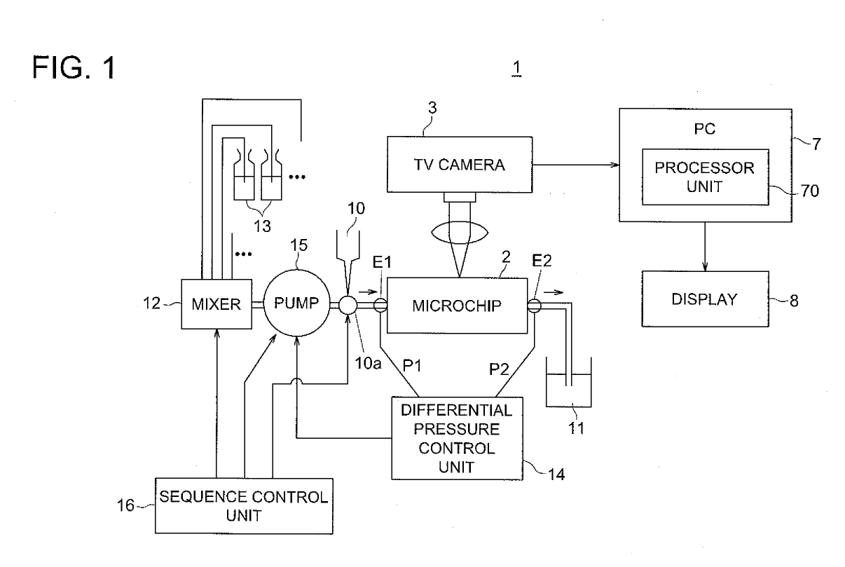

FIG. 1 is a block diagram showing a total configuration of a blood analysis system relating to the present embodiment.

As shown in this drawing, the blood analysis system 1 guides a blood from supply tank 10 through the microchip (filter) 2 to discharge tank 11, and measures blood properties from information acquired in the process.

To be more specific, blood analysis system 1 is mainly provided with microchip 2, a TV camera 3 to capture images of the blood path in microchip 2, personal computer 7 to compute the blood properties based on the blood image captured by TV camera 3, and display 8 to display the blood flow image. Blood analysis system 1 of the present embodiment is further provided with a plurality of solution bins 13, connected via mixer 12 to the blood flow, for mixing liquids such as normal saline and physiological active substance with the blood and guiding to microchip 2. Then, the blood mixed with the normal saline or the physiological active substance (hereinafter referred as blood) is made to flow in microchip 2 with a required amount, due to the adjustment of differential pressure before and after the microchip 2 performed by differential pressure control unit 14 controlling pump 15. Further, in addition to the above described mixer 12 and pump 15, bulb 10a of supply tank 10 and the like are integrally controlled by sequence control unit 16.

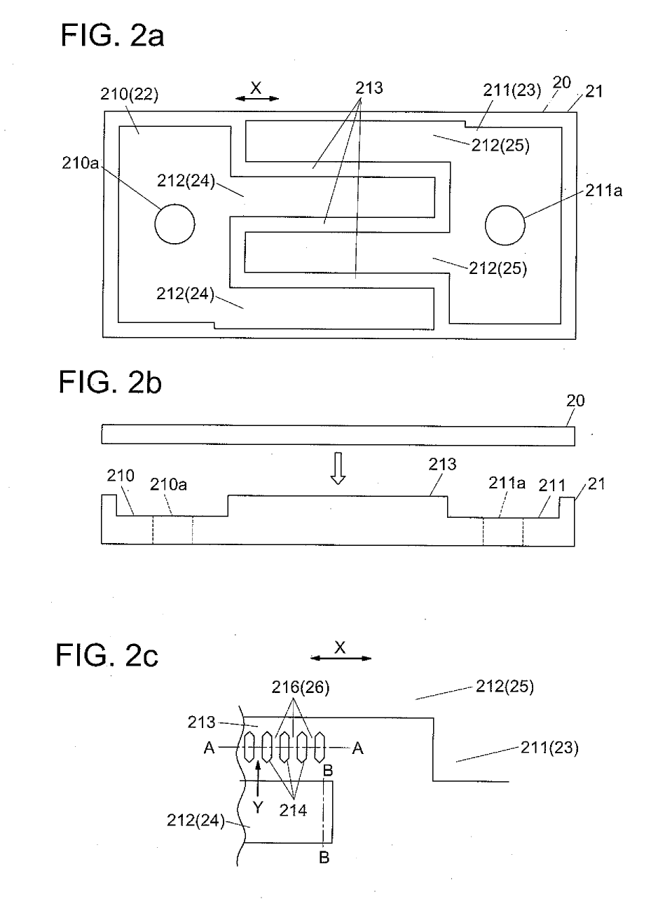

As shown in FIG. 2, microchip 2 is formed by superposing rectangular glass plate 20 and base plate 21.

Glass plate 20 is formed in flat plate shape, and covers the inside surface (upper surface in FIG. 2b) of base plate 21.

Base plate 21 is provided with depressed areas 210, 211 at both sides, and with a plurality of groove portions 212, . . . between these depressed areas 210, 211.

Depressed area 210 has pass-through slot 210a communicating with supply tank 10 at the bottom face, and forms an upstream reservoir section 22 to accumulate blood between glass plate 20 and the depressed area.

Similarly, depressed area 211 has pass-through slot 211a communicating with discharge tank 11 at the bottom face, and forms a downstream reservoir section 23 to accumulate blood between glass plate 20 and the depressed area.

Further, the plurality of groove portions 212, . . . , are disposed along in parallel to a direction (X direction in the drawing) connecting depressed area 210 and depressed area 211, and are separated by terrace portion 213 disposed along the X-direction. These plurality of groove portions 212, . . . , are alternately connected to depressed area 210 and depressed area 211 so as to form upstream blood path 24 that flows-in the blood from upstream reservoir 22 and downstream blood path 25 that flows the blood toward downstream reservoir 23, between glass plate 20 and the groove portions.

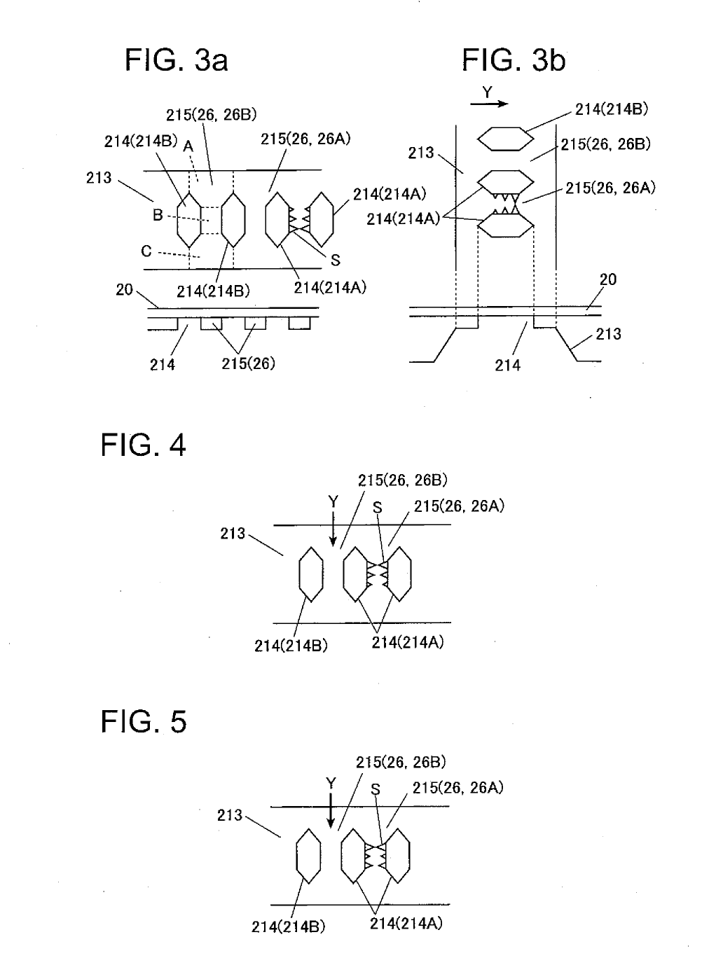

As shown in FIG. 2c and FIG. 3, at the upper-end portion of terrace portion 213, a plurality of approximately hexagonal shaped bank portions 214, . . . , are arranged in X-direction to contact glass plate 20 at the upper face.

The plurality of bank portions 214, . . . , form gate 215 between each other, and each gate 215 forms fine channel 26 to flow the blood in perpendicular direction (Y-direction) to X-direction between glass plate 20 and the gate. Further, in the present embodiment, a side face of bank portion 214 that forms channel 26 (gate 215) is arranged longitudinally to Y-direction, therefore, an inner wall portion of channel 26 (base end section of the bank portion, in a case where a barrier S described later is disposed on the bank portion) has a uniform cross section in the blood flow direction (Y-direction). Further, although not particularly restricted, the width of gate 215 is formed to be less than a blood cell diameter in the blood, for example blood cell diameter (approximately 8 mm) of a red blood cell. Further, although not particularly restricted, in cases where channel 26 is cut at a virtual line A-A in FIG. 2c, and upstream blood path 24 or down stream blood path 25 is cut at a virtual line B-B in FIG. 2c, a cross section of channel 26 is formed less than a cross section of upstream blood path 24 or down stream blood path 25. To be more in detail, the cross section shape of channel 26 is formed to be flat rectangle in accordance with the shape of red blood cell (a disk shape with concave center portion and flattened ellipsoid cross section), and the cross section size of channel 26 is made smaller than the size of red blood cell. Accordingly, a state where the red blood cell passes through a fine blood vessel such as a capillary vessel while changing its own shape can be observed, and further, degree of smooth blood flow can be reproduced in simulation.

Here, bank portion 214 and channel 26 will be described in more detail.

In the present embodiment, plurality of bank portions 214, . . . , are configured with a first bank portion 214A and a second bank portion 214B.

Among these, the first bank portion 214A is arranged to be adjacent in X-direction to one or more other first bank portion 214A. On opposing faces of each other first bank portions 214A, namely on wall faces demarcating the channel 26, a plurality of barriers (first barrier) S . . . for locally changing the direction of the blood flow are provided. These barriers S, . . . are protruded toward inside of channel 26 and are arranged in plurality along the Y-direction, and barriers S adjacent with each other have different shapes. According to this, in channel 26 existing between two first bank portions 214A (hereinafter referred as bather channel 26A), different shaped plurality of barriers S . . . are provided in the blood flow direction (Y-direction). In the present embodiment, among the side faces of first bank portion 2143, the other side face than the opposing face to the other first bank portion 214 is formed to be flat. Further, the plurality of barriers S . . . on the first bank portion 214A is arranged such that the larger barrier S is arranged toward the downstream from the upstream of the blood flow (Y-direction).

On the other hand, second bank portion 214B is arranged to oppose the other side face of the first bank portion where barrier S is provided of the first bank portion, or opposing to the side face of the other second bank portion 214B. All the side peripheral surfaces of the second bank portion are formed to be flat, accordingly, in channel 26 (hereinafter referred as comparative channel 26B) existing between of the second bank portion 214B and the other bank portion 214 (first bank portion 214A or second bank portion 214B) adjacent in X-direction, bather S is not provided, in other words, the wall faces demarcating the channel 26B are formed flat.

In microchip 2 described above, the blood supplied from supply tank 10 is accumulated in upstream reservoir 22, is passed from upstream blood path 24 through channel 26, downstream blood path 25, and after accumulated in downstream reservoir 23, the blood is discharged from discharge tank 11. To be more in detail, as shown in FIG. 3a, the blood cell such as red blood cell flowing in channel 26 firstly passes entrance area A at upstream of gate 215, passes inside area B of gate 215 while changing its shape, and finally passes exit area C at downstream of gate 215.

As a manufacturing method of such the microchip 2, for example, a method may be noted where after patterning a negative shaped mold by using such as photoresist “SU-8”, the polydimethylsiloxane or silicone is molded and de-molded by using this mold.

In the present embodiment, although every microchip is described as that made of glass for examples, the microchip may be made of resin. In that case, the base plate and/or cover plate of the microchip may be formed by mold ejection method performed by ejection of molten resin.

At front and back of the above microchip 2, pressure sensors E1, E2 are provided, and the pressure sensors E1 and E2 output each measured pressure P1, P2 to differential pressure control unit 14 (refer to FIG. 1).

TV camera 3 is, for example, a digital CCD camera which is a high speed camera having enough resolution for image-capturing the flow of blood. This TV camera 3 is instilled opposing to glass plate 20 of microchip 2, and captures images of the blood flow passing through bather channel 26A and comparative channel 26B respectively, through glass plate 20. The image-capturing area includes entrance area A to exit area C (refer to FIG. 3a) in the plurality of gates 215. However, the image-capturing area may be an area which includes at least one of entrance area A, inside area B and exit area C in each gate 215. The blood flow image having been captured by TV camera is output to PC (personal computer) 7, and displayed on display 8. The type of TV camera, not being particularly restricted, is a camera capable of movie shooting.

Personal computer 7 is connected to TV camera 3, and provided with processor unit 70 to calculate blood properties from the image information outputted from said TV camera. Processor unit 70 is an analyzing section of the present invention and analyzes the images captured at barrier channel 26A and comparative channel 26B respectively to compare the blood properties. Wherein, the blood properties are characteristic values regarding fluidity of the blood, for example, such as a velocity of blood cell and an aggregation capacity of the blood. The aggregation capacity is a quantitative value indicating generation capability of aggregation phenomena where blood cells accumulate and bind together into a mass, and the capacity is expressed with an area, a number, a ratio of area or a ratio of number of each type of blood cell contained in a blood cell accumulation section composed of accumulated blood cells. As such the processor unit 70, a conventionally known processor can be utilized.

Display 8 is connected to personal computer 7, and configured to display the captured image outputted from TV camera 3 and blood properties calculated by personal computer 7.

Subsequently, behaviors of blood analysis system in a case of measuring blood properties will be described.

Firstly, while the blood flows into microchip 2, TV camera capture images of blood flow in channel 26. To be more in detail, while supplying the blood of measuring object into supply tank 10, sequence control unit 16 controls to add normal saline solution and the like into solution bins 13 as necessary. And, while sequence control unit 16 controls to flow the blood into said microchip 2 by applying a prescribed differential pressure on microchip 2, TV camera 3 captures images of blood flows in barrier channel 26A and comparative channel 26B respectively.

Next, personal computer 7 executes image processing of the captured images and after calculating the blood properties in bather channel 26A and comparative channel 26B respectively, displays the calculation results and captured images themselves on display 8. Further, at this time personal computer 7 calculates the difference between blood properties in barrier channel 26A and blood properties in comparative channel 26B, compares both blood properties, and displays the comparison result.

As described above, according to the blood analysis system 1 of the present embodiment, among the plurality of channels 26, only on the wall face demarcating the barrier channel 26A, a plurality of barriers S for locally changing the direction of the blood flow are provided along the blood flow direction. Thus, shapes of channels are caused difference between said barrier channel 26A and comparative channel 26B. Therefore, by comparing the blood vessel having no barrier with the blood vessel having barriers, the blood properties can be accurately analyzed.

Further, since among the plurality of barriers, barriers adjacent to each other in the blood flow direction differ in shape for each other, the blood properties can be analyzed in more similar condition to that in real blood vessel, compared to the case where barriers adjacent to each other in the blood flow direction are same in shape for each other.

Therefore, accurate analysis can be performed compared to conventional method.

Further, since captured images in bather channel 26A and comparative channel 26B among the plurality of channels are analyzed respectively, and the blood properties are compared, the analysis of blood properties, for example, in a case where a thrombus or an atherosclerosis is generated in the blood vessel, can be performed.

Further, TV camera 3 captures the images of plural channels in barrier channel 26A and comparative channel 26B, the analysis of blood properties can be easily performed compared to the case where either one of the images is captured.

Further, in the above described embodiment, the plurality of barriers on the first bank portion 214A and second bank portion 214B are described such that the larger bather S is arranged toward the downstream from the upstream of the blood flow, however, the smaller barrier S may be arranged as going toward the downstream from the upstream of the blood flow. This simulates the case where there are larger sized barriers in upstream of the blood flow in the condition of blood vessel of human body, thus the condition of blood vessel in such case can be analyzed. Further, as shown in FIG. 5, in condition that the barriers adjacent to each other in the blood flow direction differ in shape for each other, the shapes may be varied irregularly. This simulates the case where size of barriers in the blood flow direction varies irregularly, thus the condition of blood vessel in such case can be analyzed.

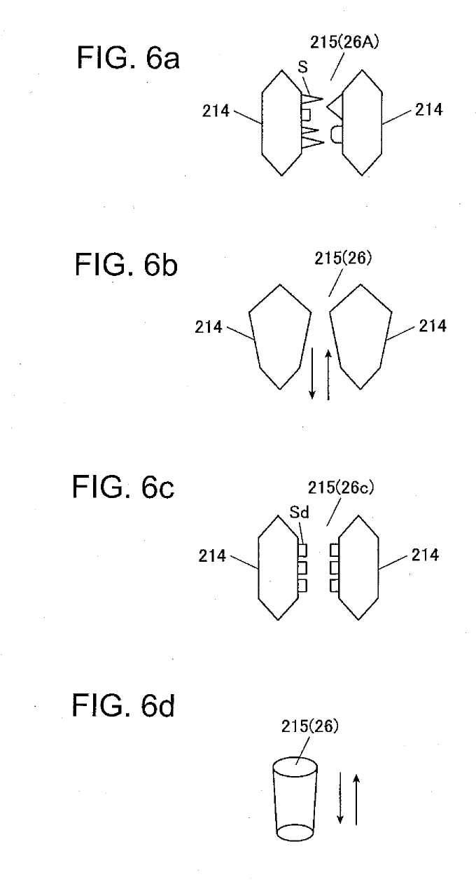

Further, although in the above description the barriers adjacent to each other in the blood flow direction differ in shape for each other, each barriers S may have a random shape as shown in FIG. 6a. In this case, the effect similar to that of the above described embodiment can be obtained.

Further, described above is that the cross section at the inner wall portion of channel 26 is uniform in the blood flow direction (Y-direction), however, the cross section may become larger toward the downstream from the upstream as shown in FIG. 6b, or may become smaller. In the case where the cross section becomes larger toward the downstream from the upstream, analysis can be performed by simulating the case of blood vessel condition of human body where the cross section becomes larger toward the downstream from the upstream. Further in the case where the cross section becomes smaller toward the downstream from the upstream, analysis can be performed by simulating the case of blood vessel condition of human body where the cross section becomes smaller toward the downstream from the upstream. In these cases, the blood properties can be analyzed by more closely simulating the flow of blood to the condition in the blood vessel, and change of the flow due to the change of blood vessel width can be analyzed.

Further, in microchip 2, the case is described where barrier channel 26A and comparative channel 26B are arranged between the plurality of bank portions 214, however, the type of channel arranged with barrier channel 26A is not limited to comparative channel 26B. For example, as shown in FIG. 6c, channel 26C where same shaped same type barriers (third barrier) Sd are arranged with constant intervals may be arranged with barrier channel 26A, or said channel 26C and comparative channel 26B are arranged together with barrier channel 26A. Further, in various channels 26 at least a part of the channels 26 being not restricted to channels with rectangular cross section, as shown in FIGS. 6b and 6d, any one of channel 26 (refer to FIG. 6b)) whose cross section becomes larger (or smaller) toward downstream from upstream, channel 26 (refer to FIG. 6d) whose cross section is circular, and channel 26 whose cross section is circular and becomes larger (or smaller) toward downstream from upstream may be utilized. Here, in the case where same shaped barriers Sd are arranged with constant intervals on the wall face demarcating the channel 26, the analysis can be performed by simulating the case where a thrombus is generated in the blood vessel, and the change of the blood flow due to the thrombus can be observed. Further, in the case where the shape of cross section of the wall face demarcating the channel 26 is made circular, the blood properties can be analyzed by more closely simulating the flow of blood to the condition in the blood vessel.

Further, the case is explained above where by analyzing the captured images in barrier channel 26A and comparative channel 26B among the plurality of channels 26, the blood properties are calculated, however, the blood channel may be calculated by analyzing a captured image in prescribed channel. In this case the analysis can be made easy.

Further according to the above description, the image capturing by TV camera and the calculation of blood properties are executed in parallel, however, by recording the blood flow images in a recording means (not illustrated) while capturing images, the calculation of blood properties may be executed after finishing all the image capturing. In this case, conditions of image capturing can be changed as necessary, and phenomena of aggregation and the like can be observed more precisely.

With respect to other points, the present invention is not restricted to the above described embodiments or variant examples, and arbitrarily changeable.

### EXPLANATION OF SIGNS

1 blood analysis system
2 microchip
3 TV camera
26 channel
26A barrier channel (subset of channels)
26B comparative channel (channel provided with no barrier)
26C channel (another subset channels)
70 processor unit (analyzing section)
S bather (first barrier, second bather)
Sd same shaped barrier (third barrier)

1. A microchip to be equipped in a blood analysis system for measuring properties of blood, comprising:
a plurality of channels through which blood passes, wherein only each of first subset channels among the plurality of channels is provided with a first plurality of barriers for locally changing a direction of blood flow, in a blood flow direction on a wall face demarcating the channels, and among the first plurality of barriers, barriers which are adjacent to each other in the blood flow direction differ in shape from each other.

2. A microchip to be equipped in a blood analysis system for measuring properties of blood, comprising:
a plurality of channels through which blood passes, wherein only each of first subset channels among the plurality of channels is provided with a second plurality of barriers for locally changing a direction of blood flow, in a blood flow direction on a wall face demarcating the channels, and the second plurality of barriers have random shapes.

3. The microchip of claim 1, wherein each of other subset channels than the first subset channels among the plurality of channels is provided with a third plurality of barriers for locally changing a direction of blood flow, in a blood flow direction on a wall face demarcating the channels with a constant interval, and the third plurality of barriers have a same shape.

4. The microchip of claim 1, wherein the plurality of barriers are provided so as to protrude inside the channels.

5. The microchip of claim 1, wherein an inner wall portion of the plurality of channels is configured to have a cross section area becoming wider or becoming narrower from upstream toward downstream of the blood flow.

6. The microchip of claim 1, wherein, the wall face demarcating the channels among the plurality of channels, on which the plurality of barriers are not provided, is formed flat.

7. The microchip of claim 1, wherein the wall face demarcating the plurality of channels has a circular section.

8. A blood analysis system comprising:
the microchip of claim 1;
an imaging device to capture an image of blood flow in the plurality of channels of the microchip; and
an analyzing section to analyze the image captured by the imaging device and calculates a blood property.

9. The blood analysis system of claim 8, wherein the analyzing section calculates the blood property by analyzing the captured image at a prescribed channel among the plurality of channels.

10. The blood analysis system of claim 8, wherein the analyzing section compares each blood properties by analyzing each of the captured images at a channel of the first subset channels and at a channel of other channels than the first subset channels among the plurality of channels respectively.

11. The blood analysis system of claim 8, wherein the imaging device captures images of blood flow at a channel of the first subset channels and at the other channel among the plurality of channels.

12. A blood analysis method utilizing the microchip of claim 1, comprising:
an imaging process to capture an image of blood flow in the plurality of channels of the microchip; and
an analyzing process to analyze the image captured by the imaging device and calculate a blood property.

13. The blood analysis method of claim 12, wherein the analyzing process calculates the blood property by analyzing the captured image at a prescribed channel among the plurality of channels.

14. The blood analysis method of claim 12, wherein the analyzing process compares each blood properties by analyzing each of the captured images at a channel of the first subset channels and at a channel of other channels than the first subset channels among the plurality of channels respectively.

15. The blood analysis method of claim 12, wherein the imaging process captures images of blood flow at a channel of the first subset channels and at a channel of other channels than the first subset channels among the plurality of channels.
